(19) United States
(12) Patent Application Publication
(10) Pub. No.: US 2026/0094269 A1
(43) Pub. Date: Apr. 2, 2026

(54) DIAGNOSIS AND MONITORING OF NEURODEGENERATIVE DISEASES

(71) Applicant: MACQUARIE UNIVERSITY, New South Wales (AU)

(72) Inventors: Dominic ROWE, New South Wales (AU); Ewa GOLDYS, New South Wales (AU); Martin GOSNELL, New South Wales (AU); Ayad ANWER, New South Wales (AU)

(21) Appl. No.: 19/414,441

(22) Filed: Dec. 10, 2025

Related U.S. Application Data
(63) Continuation of application No. 17/620,498, now abandoned.

(30) Foreign Application Priority Data
Jun. 27, 2019 (AU) 2019902257

(51) Int. Cl. G06T 7/00; A61B 5/00; G06V 20/69

(52) U.S. Cl. CPC G06T 7/0012; A61B 5/0071; A61B 5/0075; A61B 5/4082; A61B 5/4842; A61B 5/4848; G06V 20/695; G06V 20/698; G06T 2207/10036; G06T 2207/10056

(57) ABSTRACT

Disclosed is a method for diagnosing a neurodegenerative disease in a subject. The method comprises obtaining from the subject a sample comprising at least one live blood cell, and optionally isolating at least one live blood cell from the sample. The method further comprises generating one or more multispectral or hyperspectral images of the at least one cell, and analysing spectral characteristics of autofluorescence from the at least one cell. Also disclosed is a system configured to aid in the detection or diagnosis of a neurodegenerative disease. Also disclosed is a method for selecting a subject for treatment for a neurodegenerative disease. Also disclosed is a method for monitoring the response of a subject to a therapeutic treatment for a neurodegenerative disease. Also disclosed is a protocol for monitoring the efficacy of a therapeutic treatment for a neurodegenerative disease.

### CROSS-REFERENCE TO RELATED APPLICATIONS

This application is a continuation of U.S. application Ser. No. 17/620,498, filed 17 Dec. 2021, which is the US National Stage entry of International Application No. PCT/AU2020/050649, filed 26 Jun. 2020, which claims priority to Australian Patent Application No. 2019902257, filed 27 Jun. 2019.

### BACKGROUND

### Field

The present disclosure relates generally to methods for diagnosing neurodegenerative diseases, such as motor neuron diseases, and for monitoring the progression of such diseases over time, utilising hyperspectral autofluorescence imaging of live, viable cells.

### Description of Related Art

Motor neuron diseases are a group of related progressive neurodegenerative diseases affecting the motor neurons in the brain and/or spinal cord. Degeneration of the motor neurons causes muscle weakness and wastage and ultimately paralysis in many cases. Symptoms can include difficulty swallowing, limb weakness, slurred speech, impaired gait, facial weakness and muscle cramps. The nature and extent of symptoms, and progress of the diseases can differ significantly between individuals. Motor neuron diseases share an underlying pathogenesis with other neurodegenerative diseases including Parkinson's Disease and Alzheimer's Disease.

The most common form of motor neuron disease is amyotrophic lateral sclerosis (ALS), affecting motor neurons in both the brain and the spinal cord. ALS is a fatal motor neuron disease characterized by a loss of pyramidal cells in the cerebral motor cortex, anterior spinal motor neurons and brain stem motor neurons. ALS typically shows rapid deterioration after onset, often leading to death within a few years. ALS occurs in sporadic (SALS) and familial (FALS) forms, with inherited ALS accounting for less than about 10% of cases. Progressive bulbar palsy (PBP or Bulbar Onset) is a form of ALS that typically begins with difficulties in swallowing, chewing and speaking and affects approximately one quarter of ALS sufferers. Some forms of motor neuron diseases are selective in the motor neurons affected. For example, primary lateral sclerosis (PLS) affects motor neurons in the brain, while progressive muscular atrophy (PMA) affects spinal cord motor neurons.

Motor neuron diseases are debilitating, devastating and most often fatal diseases. There are approximately 2,500 sufferers in Australia, about 800 deaths per year, and about 40,000 sufferers in the United States. Percentages of death due to motor neuron diseases are increasing. Prognosis is poor and there is no cure, however new treatments are emerging that offer increased hope to sufferers in terms of prolonging life and maintaining a higher quality of life for longer. Clearly an early diagnosis is important in maximising the potential treatments available and maximising treatment benefits.

Despite much research, there remains no clear picture of the etiology of motor neuron diseases. Coupled with the fact that early symptoms may be mild and mimic those of other conditions, accurate diagnosis is challenging, in particular in the early stages. Diagnosis is typically by way of specialist neurological assessment, including magnetic resonance imagery (MRI) scans, nerve conductance tests and electromyography. While some disease biomarkers have been identified, these are typically unreliable and do not offer the potential for real time analysis and monitoring of patients.

There is a clear need for the development of simple, reliable and accurate methods for diagnosing neurodegenerative diseases such as motor neuron diseases, in particular at an early stage, and for monitoring the progress of these diseases in real time.

### SUMMARY OF THE DISCLOSURE

The present disclosure is predicated on the inventors' findings exemplified herein that hyperspectral image analysis of peripheral white blood cells can be used in the diagnosis of, and monitoring of progression and response to therapy of, motor neuron disease.

A first aspect of the present disclosure provides a method for diagnosing a neurodegenerative disease in a subject, the method comprising:
obtaining from the subject a sample comprising at least one live blood cell;
optionally isolating at least one live blood cell from the sample;
generating one or more multispectral or hyperspectral images of the at least one live blood cell; and
analysing spectral characteristics of autofluorescence from the at least one live blood cell.

Typically the spectral characteristics of autofluorescence are compared to spectral characteristics of autofluorescence from a cell(s) in or derived from one or more reference samples known to be free of the neurodegenerative disease.

The autofluorescence comprises fluorescence of one or more endogenous cellular fluorophores. The endogenous cellular fluorophores may be selected from, for example, nicotinamide dinucleotides such as nicotinamide adenine dinucleotide (NADH) and nicotinamide adenine dinucleotide phosphate (NADPH), flavins such as flavin adenine dinucleotide (FAD) and flavin mononucleotide (FMN), and porphyrins. The multispectral or hyperspectral imaging analysis may be sensitive to, or may detect or measure the content of some of these fluorophores in the at least one cell or in one or more subcellular compartments of the cell.

Typically the at least one live blood cell is a peripheral mononuclear blood cell, more typically a monocyte, lymphocyte or neutrophil. In an exemplary embodiment the at least one live blood cell is a monocyte. The method may comprise generating one or more multispectral or hyperspectral images of a multiplicity of live cells, for example a tissue or organ. In a further exemplary embodiment a suspension of monocytes in, or isolated from, the sample is subjected to the multispectral or hyperspectral autofluorescence imaging.

In a further exemplary embodiment, the sample comprising the at least one live blood cell is obtained from venous blood. Peripheral mononuclear blood cells may be prepared from the blood sample immediately after collection by isolating the buffy coat following centrifugation, optionally density gradient centrifugation. The at least one cell of interest may be isolated by negative selection.

Typically, the one or more multispectral or hyperspectral images are generated by multispectral or hyperspectral microscopy.

Typically, the step of generating one or more multispectral of hyperspectral images includes the steps of stimulating the at least one live blood cell by irradiation with electromagnetic radiation having one or more wavelengths in an excitation spectral channel and detecting autofluorescence of the at least one cell in an emission spectral channel. The step of generating one or more multispectral or hyperspectral images is typically repeated for each pair of excitation spectral channel and emission spectral channel in a set of spectral channel pairs.

Typically, the emission spectral channel differs from the excitation spectral channel.

Typically, the step of analysing spectral characteristics of autofluorescence from the cells includes the steps of: performing image pre-processing; calculating, for each cell, quantitative features of the measured autofluorescence; removing correlations between the calculated quantitative features of different cells; and projecting, for each cell, the quantitative features of the measured autofluorescence onto a new vector space. The step of removing correlations may use Principal Component Analysis (PCA). The new vector space may be produced by Linear Discriminant Analysis (LDA).

The neurodegenerative disease may be, for example, a motor neuron disease, Parkinson's disease or Alzheimer's disease. In a particular embodiment, the neurodegenerative disease is a motor neuron disease.

A second aspect of the disclosure provides a method for selecting a subject for treatment for a neurodegenerative disease, comprising:
(a) obtaining from a subject a sample comprising at least one live blood cell, and optionally isolating at least one live blood cell from the sample;
(b) executing steps of generating one or more multispectral or hyperspectral images of the at least one live blood cell, and analysing spectral characteristics of autofluorescence from the at least one live blood cell, to diagnose a neurodegenerative disease; and
(c) selecting a subject, identified in (a) as having a neurodegenerative disease, for treatment for said disease.

A third aspect of the disclosure provides a method for monitoring the response of a subject to a therapeutic treatment for a neurodegenerative disease, the method comprising:
(a) obtaining from a subject a first sample before or after commencement of therapeutic treatment, wherein the first sample comprises at least one live blood cell, and optionally isolating at least one live blood cell from the sample;
(b) executing steps of generating one or more multispectral or hyperspectral images of the at least one live blood cell from the first sample, and analysing spectral characteristics of autofluorescence from the at least one live blood cell;
(c) obtaining from the same subject a second sample at a time point after commencement of treatment and after the first sample is obtained, wherein the second sample comprises at least one live blood cell, and optionally isolating at least one live blood cell from the sample;
(d) executing steps of generating one or more multispectral or hyperspectral images of the at least one live blood cell from the second sample, and analysing spectral characteristics of autofluorescence from the at least one live blood cell; and
(e) comparing said spectral characteristics of cells from the first and second samples, wherein the comparison between said spectral characteristics between the at least one live blood cell from the first sample and the at least one live blood cell from the second sample is indicative of whether or not the subject is responding to the therapeutic treatment.

The method may further comprise obtaining and executing steps in respect of a third or subsequent sample.

A fourth aspect of the disclosure provides a protocol for monitoring the efficacy of a therapeutic treatment for a neurodegenerative disease, the protocol comprising:
(a) obtaining from a subject a first sample before or after commencement of therapeutic treatment, wherein the first sample comprises at least one live blood cell, and optionally isolating at least one live blood cell from the sample;
(b) executing steps of generating one or more multispectral or hyperspectral images of the at least one live blood cell from the first sample, and analysing spectral characteristics of autofluorescence from the at least one live blood cell;
(c) obtaining from the same subject a second sample at a time point after commencement of treatment and after the first sample is obtained, wherein the second sample comprises at least one live blood cell, and optionally isolating at least one live blood cell from the sample;
(d) executing steps of generating one or more multispectral or hyperspectral images of the at least one live blood cell from the second sample, and analysing spectral characteristics of autofluorescence from the at least one live blood cell; and
(e) comparing said spectral characteristics of cells from the first and second samples, wherein the comparison between said spectral characteristics between the at least one live blood cell from the first sample and the at least one live blood cell from the second sample is indicative of whether or not the therapeutic treatment is effective.

The protocol may further comprise obtaining and executing steps in respect of a third or subsequent sample.

The protocol may also be used in the screening of candidate agents for treating the neurodegenerative disease.

A fifth aspect of the disclosure provides a system configured to aid in the detection or diagnosis of a neurodegenerative disease, the system including: a light source for stimulating live blood cells by irradiation with electromagnetic radiation having one or more wavelengths in an excitation spectral channel; a detector for detecting autofluorescence of the cells; and a processing system configured to analyse spectral characteristics of autofluorescence of the cells, and optionally to provide a diagnostic prediction with respect to a subject.

In an embodiment, the processing system is further configured to:
perform image pre-processing;
calculate, for each cell, quantitative features of the measured autofluorescence;
remove correlations between the calculated quantitative features of different cells; and
project, for each cell, the quantitative features of the measured autofluorescence onto a new vector space.

### BRIEF DESCRIPTION OF THE DRAWINGS

Embodiments of the disclosure are described herein, by way of non-limiting example only, with reference to the following figures.

FIG. 1 illustrates an example method of spectral analysis of cells.

FIG. 2 illustrates an example method of obtaining one or more multispectral images of cells.

FIG. 3 illustrates an example method of analysing spectral characteristics of fluorescence of cells.

FIG. 4 is a scatter plot of data derived from measured fluorescence of cells from multiple subjects. Control data C1, C2, C3, and C4 is from monocytes isolated from individuals known not to have motor neuron disease (i.e. a control group); Data P1 to P15 is from a patient group. Data P1, P2, and P6 is from individual LJ during treatment with CuATSM; Data P3 is from individual RB treated with Riluzole (brand name—Rilutek™); Data P4 is from individual EG treated with Riluzole (brand name—Rilutek™); Data P5, P10, P13, and P15 is from individual JH during treatment with Riluzole (brand name—Rilutek™) and CuATSM; Data P14 is from individual WG treated with Riluzole/Abamune (anti-HIV medication); Data P7 is from individual BL treated with Riluzole (brand name—Rilutek™); Data P8 is from individual RM treated with Riluzole (brand name—Rilutek™).

FIG. 5 is a scatter plot of data derived from measured fluorescence of cells from multiple subjects, showing clustering of monocytes. FIG. 5(a) shows data for two patients and one control individual, including data from Patient 1 in an ALS region (prior to treatment) and in a healthy region (following treatment). The axes represent an optimised combinations of spectral channels. FIG. 5(b) shows response scores for two patients with uncontrolled disease before they entered the CuATSM clinical trial (timepoint TO) and four control individuals. FIG. 5(c) shows longitudinal testing of Patients 1 and 2 at consecutive time points (T1-T3). Each point on the scatter plot corresponds to data from an: individual cell.

FIG. 6 is a scatter plot showing response scores for eight ALS patients undergoing treatment with different drugs, as indicated in the patient listing provided above for FIG. 4.

FIG. 7 illustrates an example system for spectral analysis of cells.

FIG. 8 illustrates an example processing system for use in the system of FIG. 7.

### DETAILED DESCRIPTION OF A PREFERRED EMBODIMENT

Unless defined otherwise, all technical and scientific terms used herein have the same meaning as is commonly understood by one of skill in the art to which the disclosure belongs.

As used herein, the singular forms “a”, “an” and “the” also include plural aspects (i.e. at least one or more than one) unless the context clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” means originates from or obtained from. The terms “derived from” and “obtained from” may be used interchangeably herein.

As used herein, the term “negative selection” refers to any method of selecting one or more desired cells by depletion of all cells except desired cells, thereby leaving the one or more desired cells ‘untouched’ and ‘unaffected’ (e.g. unlabelled). Unwanted cells are typically depleted by labelling or binding with a suitable moiety (e.g. an antibody) to facilitate removal of the unwanted cells. Those skilled in the art will appreciate that negative selection as defined and contemplated herein is distinguished from positive selection in which desired cells are actively selected by labelling or binding with a suitable moiety thereby facilitating isolation.

The term “subject” as used herein refers to mammals and includes humans, primates, livestock animals (e.g. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Typically, the mammal is human or a laboratory test animal. More typically, the mammal is a human.

Multispectral and hyperspectral imaging uses colour and spatial image information for detection and classification. In the methods of the present disclosure autofluorescence images of live cells are obtained at a number of selected excitation wavelength ranges, capturing their emission at multiple specified wavelength ranges. This accurately quantifies their (autofluorescence) colour.

Pairs of excitation and emission channels with selected excitation wavelength range and emission wavelength range have been used in all hyperspectral or multispectral imaging described here, as per Table in paragraph [0149]. Alternative spectral channels can be used, but, in some examples, these should cover a sufficient portion of UV and short visible electromagnetic spectrum.

### Methods and Diagnostic Tests

The present disclosure relates to the inventors' application of multispectral and hyperspectral autofluorescence imaging of circulating peripheral mononuclear blood cells to detect and diagnose neurodegenerative disease. Accordingly, the present disclosure provides, for the first time, a reliable, accurate blood-based diagnostic test for detecting neurodegenerative disease. This diagnostic test opens the possibility of diagnosing neurodegenerative diseases prior to the onset or manifestation of clinical symptoms, in turn allowing treatment to be commenced in this preclinical window. The ability to commence early treatment can be crucial in improving patient prognosis, maintaining or extending quality of life, or in delaying or preventing the onset of clinical symptoms of the disease.

The disclosure also provides real time assays for monitoring the response of a subject to a treatment for a neurodegenerative disease, and for determining the efficacy of a treatment for a neurodegenerative disease in a specific subject. For example, the methods may be used to evaluate the efficacy of a new therapeutic agent or treatment regime or protocol, or may be used in the context of personalised medicine to determine if a specific individual will respond to a specific treatment.

One aspect of the present disclosure provides a method for diagnosing a neurodegenerative disease in a subject, the method comprising:
obtaining from the subject a sample comprising at least one live blood cell;
optionally isolating at least one live blood cell from the sample;
generating one or more multispectral or hyperspectral images of the at least one live blood cell; and
analysing spectral characteristics of autofluorescence from the at least one live blood cell.

Methods of the present disclosure analyse spectral images from live, viable blood cells. While exemplified herein with the spectral analysis of autofluorescence from monocytes, the methods may be employed using other peripheral mononuclear blood cells such as lymphocytes and neutrophils. Samples comprising cells to be analysed may be venous blood samples. Blood samples may be obtained using standard methods known to those skilled in the art. Blood may be used immediately or may be stored under suitable conditions until required, provided the storage maintains the viability of one or more blood cells to be isolated from the sample for analysis. In particular embodiments live blood cells for spectral analysis are isolated from fresh blood samples, thus enabling real time testing to be undertaken in accordance with methods of the present disclosure.

A number of techniques well known to those skilled in the art may be used for isolation of live cells. As exemplified herein, peripheral mononuclear blood cells may be isolated from the buffy coat of a blood sample following centrifugation, optionally density gradient centrifugation, such as a Ficoll or Ficoll-Paque gradient. The cells to be subjected to spectral analysis may then be separated from other peripheral blood mononuclear cells by negative selection. By way of example only, typically unwanted cells are labelled, for example magnetically labelled using one or more biotin or other suitably conjugated antibodies against cell surface molecules and/or microbeads. Isolation of a highly pure population of unlabelled cells of interest is then achieved by depletion of the labelled unwanted cells (negative selection). Negative selection offers the advantage that the cells of interest are unlabelled and untouched, have not been exposed to a column or other stressors, and are therefore of high viability. In the case of immune cells it may be critical that they have not been stimulated by the process of selection. Suitable methods and protocols for the isolation of pure, live, viable cells, including methods and protocols for negative selection, will be well known to those skilled in the art.

The methods of the present disclosure are amenable to the diagnosis of a range of neurodegenerative diseases, including for example motor neuron diseases, Parkinson's disease including idiopathic Parkinson's disease and familial monogenic Parkinson's disease, Alzheimer's disease or frontotemporal dementia. In particular embodiments, the neurodegenerative disease is a motor neuron disease. The motor neuron disease may be, for example, amyotrophic lateral sclerosis (ALS), primary lateral sclerosis (PLS) or progressive muscular atrophy (PMA).

Methods of the present disclosure detect and measure autofluorescence, that is cellular fluorescence from one or more endogenous cellular fluorophores. The endogenous cellular fluorophores may be, for example, nicotinamide dinucleotides such as nicotinamide adenine dinucleotide (NADH) and nicotinamide adenine dinucleotide phosphate (NADPH), flavins such as flavin adenine dinucleotide (FAD) and flavin mononucleotide (FMN), porphyrins, elastin, collagen, tryptophan and pyridoxine.

Without wishing to be bound by theory, the inventors suggest that the present methods detect metabolic changes in the cells analysed, in particular in peripheral mononuclear blood cells, relating to the redox state in the cells, for example determined by inflammation and/or mitochondrial changes.

Typically, the spectral characteristics of autofluorescence in cells isolated from the subject under assessment are compared to spectral characteristics of autofluorescence from one or more cells isolated or derived from one or more reference samples known to be free of the neurodegenerative disease. In this context the term “reference” or “reference sample” means one or more biological samples from individuals or groups of individuals diagnosed as not having neurodegenerative disease. A “reference sample” may therefore comprise the compilation of data from one or more individuals whose diagnosis as a “reference” or “control” for the purposes of the present disclosure has been confirmed. That is, samples to be used as reference samples or controls need not be specifically or immediately obtained for the purpose of comparison with the sample(s) obtained from a subject under assessment.

Methods of the present disclosure may be employed to detect or diagnose a neurodegenerative disease in a subject where no diagnosis, or confirmed diagnosis, previously existed. Such diagnosis may be made in the absence of clinical symptoms of the disease. For example, a subject may present as having an increased risk of, or otherwise susceptible to, the development of a neurodegenerative disease, for example as a result of family history. Alternatively, the methods disclosed herein may be used to confirm a diagnosis or preliminary diagnosis offered by a different means, for example, MRI scans, nerve conductance tests, electromyography, other neurological assessments, or one or more biomarkers of the disease. Thus, the present methods may be used independently, or in conjunction, with one or more other diagnostic methods, tests or assays.

A subject identified, in accordance with the methods of the present disclosure described hereinbefore as having a neurological disease, can be selected for treatment, or stratified into a treatment group, wherein an appropriate therapeutic regimen can be adopted or prescribed with a view to treating the disease.

Thus, in an embodiment, the methods disclosed herein may comprise the step of exposing (i.e., subjecting) a subject identified as having a neurodegenerative disease to a therapeutic treatment or regimen for treating said disease. The nature of the therapeutic treatment or regimen to be employed can be determined by persons skilled in the art and will typically depend on factors such as, but not limited to, the age, weight and general health of the subject.

An aspect of the disclosure therefore provides a method for selecting a subject for treatment for a neurodegenerative disease, comprising:
(a) obtaining from a subject a sample comprising at least one live blood cell and optionally isolating at least one live blood cell from the sample;
(b) executing steps of generating one or more multispectral or hyperspectral images of the at least one live blood cell, and analysing spectral characteristics of autofluorescence from the at least one live blood cell, to diagnose a neurodegenerative disease; and
(c) selecting a subject, identified in (a) as having a neurodegenerative disease, for treatment for said disease.

As used herein the terms “treating” and “treatment” refer to any and all uses which remedy a neurodegenerative disease or one or more symptoms thereof, or otherwise prevent, hinder, retard, or reverse the progression of the neurodegenerative disease or one or more symptoms thereof in any way whatsoever. Thus the term “treating” and the like are to be considered in their broadest context. For example, treatment does not necessarily imply that a patient is treated until total recovery. In conditions which display or a characterized by multiple symptoms, the treatment or prevention need not necessarily remedy, prevent, hinder, retard, or reverse all of said symptoms, but may prevent, hinder, retard, or reverse one or more of said symptoms.

It will be clear to the skilled addressee that the methods disclosed herein can be also used to monitor the response of a subject to, and the efficacy of, treatment of a neurodegenerative disease, whereby the spectral characteristics of autofluorescence according to the above described methods may be determined based on multispectral or hyperspectral imaging of blood cells, typically peripheral mononuclear blood cells isolated from the subject at two or more separate time points, optionally including before commencement of treatment, during the course of treatment and after cessation of treatment, to determine whether said treatment is effective.

Thus, the disclosure provides a method for monitoring the response of a subject to a therapeutic treatment for a neurodegenerative disease, the method comprising:
(a) obtaining from a subject a first sample before or after commencement of therapeutic treatment, wherein the first sample comprises at least one live blood cell, and optionally isolating at least one live blood cell from the sample;
(b) executing steps of generating one or more multispectral or hyperspectral images of the at least one live blood cell from the first sample, and analysing spectral characteristics of autofluorescence from the at least one live blood cell;
(c) obtaining from the same subject a second sample at a time point after commencement of treatment and after the first sample is obtained, wherein the second sample comprises at least one live blood cell, and optionally isolating at least one live blood cell from the sample;
(d) executing steps of generating one or more multispectral or hyperspectral images of the at least one live blood cell from the second sample, and analysing spectral characteristics of autofluorescence from the at least one live blood cell; and
(e) comparing said spectral characteristics of cells from the first and second samples, wherein the comparison between said spectral characteristics between the at least one live blood cell from the first sample and the at least one live blood cell from the second sample is indicative of whether or not the subject is responding to the therapeutic treatment.

Also provided is a protocol for monitoring the efficacy of a therapeutic treatment for a neurodegenerative disease, the protocol comprising:
(a) obtaining from a subject a first sample before or after commencement of therapeutic treatment, wherein the first sample comprises at least one live blood cell, and optionally isolating at least one live blood cell from the sample;
(b) executing steps of generating one or more multispectral or hyperspectral images of the at least one live blood cell from the first sample, and analysing spectral characteristics of autofluorescence from the at least one live blood cell;
(c) obtaining from the same subject a second sample at a time point after commencement of treatment and after the first sample is obtained, wherein the second sample comprises at least one live blood cell, and optionally isolating at least one live blood cell from the sample;
(d) executing steps of generating one or more multispectral or hyperspectral images of the at least one live blood cell from the second sample, and analysing spectral characteristics of autofluorescence from the at least one live blood cell; and
(e) comparing said spectral characteristics of cells from the first and second samples, wherein the comparison between said spectral characteristics between the at least one live blood cell from the first sample and the at least one live blood cell from the second sample is indicative of whether or not the therapeutic treatment is effective.

The above method or protocol may further comprise obtaining and executing steps in respect of a third or subsequent sample. A change in spectral characteristics of autofluorescence between cells from the first and second (or subsequent) sample may be indicative of an effective therapeutic treatment or regimen and positive response of the subject to a treatment. Where the method or protocol indicates that the therapeutic treatment or regimen is ineffective and/or the subject is not responding sufficiently to the treatment (i.e. no or insignificant change in spectral characteristics), the method or protocol may further comprise altering or otherwise modifying the therapeutic treatment or regimen with a view to providing a more efficacious or aggressive treatment. This may comprise administering to the subject additional doses of the same agent with which they are being treated or changing the dose and/or type of medication or other treatment.

Those skilled in the art will appreciate that the methods described herein, and diagnostic tests embodying such methods, may be practiced or provided via a variety of means. For example, diagnostic tests may be provided as a laboratory service, for example by pathology services or may be provided in a suitable kit or device as a as a point of care test or system, for example in the form of a handheld device. Those skilled in the art will appreciate that the scope of the present disclosure is not limited by reference to the means by which methods, tests, assay, kits, devices and systems of the present disclosure are provided.

### Multispectral and Hyperspectral Imaging and Systems of the Disclosure

The following modes, given by way of example only, are described in order to provide a more precise understanding of the subject matter of exemplary or typical embodiments. In the figures, incorporated to illustrate features of exemplary embodiments, like reference numerals are used to identify like parts throughout the figures.

To the extent that a method or individual steps of a method is/are described in this description, the method or individual steps of the method can be executed by an appropriately configured system and/or an individual device of the system. Analogous remarks apply to the elucidation of the operation mode of a system and/or individual devices of the system that execute(s) method steps. To this extent, apparatus features and method features of this description are equivalent.

A multispectral image is an image that captures image data within specific wavelength ranges in the electromagnetic spectrum. Typically, though not necessarily, the one or more multispectral images are obtained by multispectral microscopy. In other examples, the one or more multispectral images are obtained by hyperspectral microscopy. In yet other examples, the one or more multispectral images are obtained by any other multispectral or hyperspectral imaging method.

Hyperspectral imaging uses wavelength and spatial image information for detection and classification. In one example, fluorescence images of live cells/tissues are obtained at a number of selected excitation wavelength ranges (referred to here as excitation channels), capturing their emission at multiple specified wavelength ranges (referred to as emission channels). By using optical microscopy techniques, the autofluorescence of endogenous cellular fluorophores can be observed at a single cell level providing insights into cell activities without altering them with exogenous labels.

The terms “multispectral” and “hyperspectral” are used interchangeably here. The term “multispectral” generally refers to cases where the number of excitation or emission channels is, for example, 10 or less. The term “hyperspectral” generally refers to cases where the number of excitation or emission channels is, for example, in the order of 100 or more. Typically, mathematical analysis of data acquired through multispectral or hyperspectral microscopy is identical.

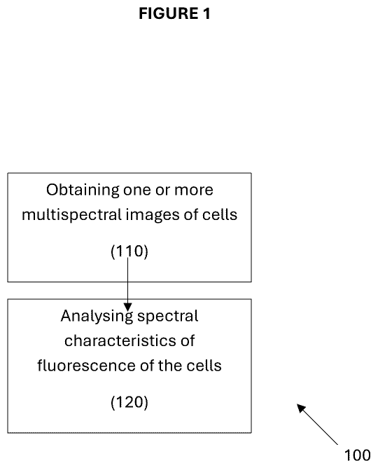

Referring to FIG. 1, there is illustrated an example method 100 of spectral analysis of cells. Method 100 includes step 110 of obtaining one or more multispectral images of cells. Method 100 further includes step 120 of analysing spectral characteristics of fluorescence of the cells. Step 120 of analysing spectral characteristics of fluorescence of the cells may include performing bioinformatics analysis.

In some examples, step 110 involves taking N images of cells (each image corresponding to one of N different excitation spectral channels), where each image includes M pixels. Therefore, for each pixel, N separate spectra are measured, each spectrum comprising fluorescence intensities due to one of the N excitation spectral channels.

In some examples, the aim of step 120 is to separate the cells into distinct classes. Examples of classes may include cells with low mutational loads (e.g. healthy cells), cells with high mutational loads (e.g. diseased cells), and corresponding control cells (i.e. cells with a known mutational load). Step 120 of analysing spectral characteristics of fluorescence of the cells may include one or more steps, described below.

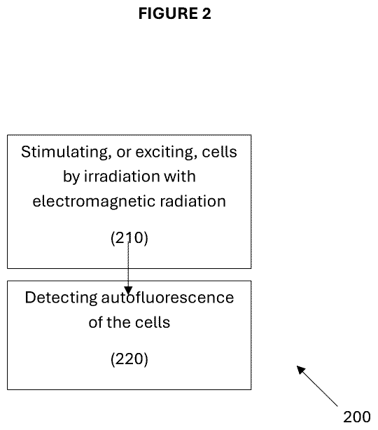

Referring to FIG. 2, there is illustrated an example method 200 of obtaining one or more multispectral images of the cells. Method 200 includes step 210 of stimulating, or exciting, the cells by irradiation with electromagnetic radiation having one or more wavelengths in an excitation spectral channel, followed by step 220 of detecting autofluorescence of the cells in spectral ranges defined by emission spectral channels.

Step 210 may be repeated for each excitation spectral channel in a set of excitation spectral channels. For each excitation spectral channel, autofluorescence of the cells may be detected in an emission spectral channel of a set of emission spectral channels. In this way, each image of the one or more multispectral images corresponds to a specific pair of excitation and emission spectral channels. Typically, though not necessarily, the images are taken in rapid succession, so as to minimise variations in the cells between images. Typically, though not necessarily, the images are taken using different fields of view on the same cell sample. For reference, in some examples, an exposure time for each image may vary between a fraction of a second and tens of seconds. In some examples, the exposure time is approximately 1 second. Such exposure times are typical for excitation powers at the objective at the few microwatts range and they depend on the values of these excitation powers, and on the imaging cameras used and their settings, such as gain.

A spectral channel defines a spectral range, or a spectral band, including one wavelength or multiple wavelengths. In some examples, the excitation radiation has a spectral profile of finite, non-zero linewidth, centred at a central excitation wavelength. For example, the excitation radiation may have a 10 nm linewidth centred at an excitation wavelength of 334 nm. In some examples, the excitation radiation has a spectral profile including a single excitation wavelength, or a very narrow wavelength range, such as less than 1 nm when the excitation wavelength is generated by a laser. In some examples, the excitation radiation has a range of wavelengths, or a plurality of wavelengths, within the excitation spectral channel, or spectral band.

Step 220 of detecting autofluorescence of the cells detects fluorescence at a predetermined detection wavelength range. In some examples, the step of detecting autofluorescence of the cells detects fluorescence at a predetermined emission channel or band, corresponding to a range of detection wavelengths. In some examples, the step of detecting autofluorescence of the cells detects autofluorescence in an emission spectral channel whose wavelength range differs from the wavelength range in the excitation spectral channel. The difference between the excitation and emission spectral channels arises because fluorescence generally occurs at wavelengths longer than the excitation wavelength. The emission spectral channel may correspond to a predetermined autofluorescence emission range.

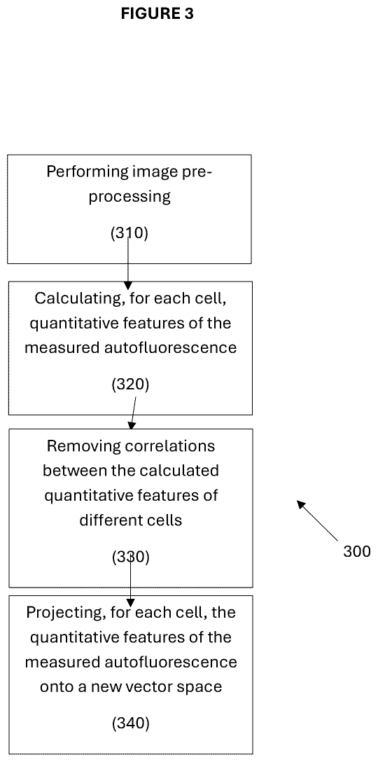

Referring to FIG. 3, there is illustrated an example method 300 of analysing spectral characteristics of autofluorescence of the cells. Method 300 includes step 310 of performing image pre-processing. Step 310 may comprise steps A to J, described below. Method 300 further includes step 320 of calculating, for each cell, a set of quantitative features of the measured autofluorescence using the information from some or all pairs of excitation and emission channels where images have been taken. Method 300 further includes step 330 of feature decorrelation (by PCA) and, and step 340 of projecting, for each cell, the decorrelated quantitative cellular feature vectors (one for each segmented cell) onto a new vector space. The feature vectors are explained below.

The multispectral/hyperspectral quantitative features of cells may be represented as vectors in a multi-dimensional vector space whose coordinates are values of these cellular features (one coordinate per feature). In some cases, these features are calculated using cellular data in only a single pair of excitation/emission channels, such as average intensity, entropy, kurtosis and many others. In other cases, these quantitative features may be calculated from several of such pairs, such as for example channel ratios, or average abundance of unmixed individual fluorophores, such as NADH, FAD etc. This vector space is referred to as “feature space” and cellular data are represented as vectors in this space, one vector per each segmented cell.

The step of removing correlations uses Principal Component Analysis. The use of a pre-processing PCA step is one way to avoid numerical problems in the later LDA stage when calculating a within-group sum of squares and cross products matrix which turns out to be singular if the input variables are linearly correlated. This PCA stage is unsupervised, and it uses the covariance matrix derived from all of the calculated feature vectors.

PCA is necessary to produce a decorrelated version of the feature data, and this prevents numerical problems in later stages of the analysis. This decorrelated version of data leaves the important information about cell differences whilst removing data correlations. This procedure transforms the original basis vectors in the feature space into specific new basis vectors that are rotated with respect to the original basis vectors. The original dataset is transformed in such a way that the features become maximally decorrelated. This dataset still retains the meaning and the information content of the original features. Further data analysis proceeds in this new PCA-decorrelated space.

At step 320, the example quantitative feature may be a spatial average of channel intensity for each cell divided by the cell area for each cell. To calculate this example quantitative feature, the pixels corresponding to a specific cell may be identified by a procedure known as “cell segmentation”, and the corresponding autofluorescence signal measured within those specific pixels added across each pixel, separately for each pair of the excitation spectral channel and emission spectral channel used. The area of the cell is then calculated by counting the pixels belonging to that cell. The two values thus obtained are then divided producing average autofluorescence intensity in that cell in this pair of excitation/emission spectral channels. In other examples, the quantitative feature may be a mean, median, variance, kurtosis, or other Haralick feature calculated for each channel separately, or various derivative features combining individual channel features, such as channel ratios (the ratio of average cell intensities in two different channels) derived from the measured multispectral or hyperspectral autofluorescence images for that specific cell. These features are separately calculated in all pairs of excitation/emission spectral channels. The features may be defined in a way that reflects specific biology of the cell under investigation, for example average content of specific fluorophores of relevance to a particular cellular pathway (such as bound NADH, free NADH, FAD, flavins, cytochrome C and many others), or for example characteristics of the mitochondria such as their perinuclear location or specific shape, or shape distribution. Then the average channel intensity for each cell, or any other quantitative channel feature or the set of features for each cell, may be assigned a type or class label such as “healthy”, “reference” or “diseased” or alternatives.

It may also be possible to use non-cellular features for classification, in particular pixel features, where features for each cell are not calculated, but the pixel data for each of the classes or groups are used. For example, one could use raw pixel autofluorescence signals or secondary features such as width of pixel autofluorescence signal distributions in each of the channels as quantitative features. Alternatively, one could use quantitative features which incorporate some cellular identifications but do not imply taking cellular averages such as, for example, raw pixel autofluorescence signals, only from the largest 10% of cells.

The feature space may then be transformed to a “new vector space” as per paragraph below to optimally present cell group separation.

In some examples, the new vector space is produced by Linear Discriminant Analysis (see, for example, J. Ye, “Characterisation of a family of algorithms for generalized discriminant analysis on undersampled problems”, J. Mach. Learn. Res. 6 (2005) 483-502). The “new” vector space means a vector space whose set of basis vectors differs from the set of basis vectors of the original feature space. In other examples, the new vector space may be produced by alternative methods, including rotation under subjective manual control.

This means that the set of quantitative features thus obtained for each cell can then be projected, at step 340, onto a vector space that optimally discriminates or separates the data based on this class assignment. This projection may be done by using LDA. The dimensionality of this new space and hence the number of new canonical variables is P−1, where P is the number of unique classes assigned to the data (for example, if trying to separate two classes “healthy” and “sick”, then P=2 and the projection is onto a 1 dimensional space). The data are projected onto specific directions determined by LDA, these directions based on the actual cell data. The coordinates of each cell are now expressed in terms of these canonical variables, sometimes called “spectral variables”, and reflect the distance measured along these specific directions. Two out of P−1 directions may be selected to generate scatterplots to aid in visualising the data.

Therefore, in method 300, P cell classes are initially chosen and, by using LDA, the original feature space and the vectors representing the cell features are projected onto a new, lower dimensional space. Its dimension is given by the number of groups of cell classes to be distinguished, less 1. In some examples, three classes of cells may be used, so that after LDA the spectra of these cells can be depicted as points on two-dimensional plots. This two-dimensional spectral space produced by LDA is one of the examples of “canonical spectral spaces” that are convenient for visualisation. Its basis vectors are orthogonal, and may be aligned with the axes in two-dimensional plots for visual representation.

The LDA method ensures that the new space is optimised to provide the best degree of separation between selected cell classes (such as, for example, cells from different patients). In some examples, in order to quantify the distinctiveness between selected pairs of cell clusters, the LDA analysis may be performed again on each pair of cell cluster data projecting them onto a one-dimensional line. The Kolmogorov-Smirnov or alternative statistical tests such as t-test may then be applied to gauge and compare the similarity of the pair of clusters. In some examples, the maximum Fisher statistical distance may also be calculated. This is a measure of cluster closeness which is sensitive to cluster means and takes account of the data dispersion.

In some examples, data for additional cells, cell groups, and/or patients may be plotted together with the previously obtained cell autofluorescence data as transformed by method 300. In this approach the new data are projected on the vector space optimised to provide best separation of the original groups, but not necessarily the new groups formed by integrating the previous groups with new data. Although there is no mathematical certainty that optimum separation will be achieved for such blended datasets, a clear separation may often be achieved in the case when class distinction results are statistically strong with small p-values.

Bioinformatics analysis as used in method 100 may identify one or more quantitative features of the cells which, in combination, enable distinguishing cell ensembles of healthy patients from those of diseased patients.

Pixel intensity values are defined for a given excitation spectral channel by the measured autofluorescence intensity at each pixel in the hyperspectral/multispectral image. Using these values, and having segmented the cells, it is possible to calculate quantitative cellular features such as mean, median, variance, kurtosis, or other features. It is important for some of these features to be divided by the cell area calculated, for example as the number of pixels belonging to that cells. Therefore, a set of quantitative features may be calculated on the basis of pixel intensities for each cell captured by the hyperspectral/multispectral image.

Pixel intensity ratios are defined for a given pair of excitation spectral channels by the ratio of the measured autofluorescence intensity at each pixel of the first pair of excitation/emission channels with respect to the measured autofluorescence intensity at each pixel of the second pair of excitation/emission channels. Using these vectors for each specific cell, it is possible to calculate quantitative features such as mean, median, variance, kurtosis, or other quantitative features. Therefore, a multitude of quantitative features derived from pixel intensity ratios may be calculated for each cell captured by the hyperspectral/multispectral image. In analogy to this example, more involved pixel functions of alternative types may also be calculated, and related cellular features produced.

The calculated quantitative feature vectors for each cell may be arranged in a P by Q matrix, where P is the number of cells and Q is the number of quantitative features for each cell. The data may then undergo further processing, by PCA or LDA or alternatives as described above, causing the new variables to satisfy the group variance maximisation criteria.

The uncorrelated variables (post-PCA) can further be used for discriminatory analysis. The discriminatory analysis provides another set of variables maximising the separation between pre-specified groups. The number of variables returned by PCA and discriminatory analysis is equal to the number of statistical features, however, in some examples, only some of the variables may be plotted for data visualisation.

The use of a pre-processing PCA step is one way to avoid numerical problems in the later LDA stage when calculating a within-group sum of squares and cross products matrix which turns out to be singular if the input variables are linearly correlated. This PCA stage is unsupervised, while the LDA stage uses prior knowledge of class assignment through data labelling and attempts to find a projection that optimally separates the data based on second order statistics through the use of Fishers statistical distance criterion.

The measured feature set for each cell (being a vector) can then be projected, at step 340, into a vector space that optimally discriminates or separates the data based on the class assignment (e.g sick vs healthy). This projection may be done by using LDA. The dimensionality of this new space and hence the number of new canonical variables is P−1, where P is the number of unique classes assigned to the data. The data are projected onto specific directions determined by LDA and based on the actual cell data. The coordinates of each cell are now expressed in terms of these canonical variables, in this case called “spectral variables”, the distance measured along these specific directions. Two out of P−1 directions may be selected to generate scatterplots to aid in visualising the data.

Therefore, in method 300, P cell classes are initially chosen (e.g, “sick” “healthy”, “treated with drug X”, “treated with drug Y” etc) and, by using LDA, the original N-dimensional feature space and the data points representing the feature vectors of the cells are projected onto a new, lower dimensional space. Its dimension is given by the number of groups of cell classes to be distinguished (P), less 1. In some examples, three classes of cells may be used, so that after LDA the spectra of these cells can be depicted as points on two-dimensional plots. This two-dimensional spectral space produced by LDA is one of the examples of “canonical spectral spaces” that are convenient for visualisation. Its basis vectors are orthogonal, and may be aligned with the axes in two-dimensional plots for visual representation.

The LDA method ensures that the new space is optimised to provide the best degree of separation between selected cell classes (such as, for example, cells from different patients). In some examples, in order to quantify the distinctiveness between selected pairs of cell clusters, the LDA analysis may be performed again on each pair of cell cluster data projecting them onto a one-dimensional line. The Kolmogorov-Smirnov or alternative statistical tests such as t-test may then be applied to gauge and compare the similarity of the pair of clusters. In some examples, the maximum Fisher statistical distance may also be calculated. This is a measure of cluster closeness which is sensitive to cluster means and takes account of the data dispersion.

In some examples, data for additional cells, cell groups, and/or patients may be plotted together with the previously obtained cell autofluorescence data as transformed by method 300. In this approach the new data are projected on the vector space optimised to provide best separation of the original groups, but not necessarily the new groups formed by integrating the previous groups with new data. Although there is no mathematical certainty that optimum separation will be achieved for such blended datasets, a clear separation may often be achieved in the case when class distinction results are statistically strong with small p-values.

Bioinformatics analysis as used in method 100 may identify one or more quantitative features of the cells which, in combination, enable distinguishing cell ensembles of healthy patients from those of diseased patients.

Quantitative analysis of spectral characteristics of the cells before the cellular features can be calculated requires a step of performing image pre-processing. The steps of the method may include steps A to J described in Example 3 below. As previously indicated, the method further includes a step of calculating, for each cell, a set of quantitative features of the measured autofluorescence using the information from some or all pairs of excitation and emission channels where images have been taken. The method further includes steps of feature decorrelation (by PCA) and projecting, for each cell, the quantitative cellular feature vectors (one for each segmented cell) onto a new vector space. The projection may be such as to ensure optimal separation of the examined cell groups, for example, cells from each patient and control healthy patients. The projection may be obtained by using LDA.

The multispectral/hyperspectral quantitative features of cells may be represented as vectors in a multi-dimensional vector space whose coordinates are values of these cellular features (one coordinate per feature). In some cases, these features are calculated using cellular data in only a single pair of excitation/emission channels, such as average intensity, entropy, kurtosis and many others. In other cases, these quantitative features may be calculated from several of such pairs, such as for example channel ratios, or average abundance of unmixed individual fluorophores, such as NADH, FAD etc. This vector space is referred to as “feature space” and cellular data are represented as vectors in this space, one vector per each segmented cell.

The step of removing feature correlations uses Principal Component Analysis. The use of a pre-processing PCA step is one way to avoid numerical problems in the later LDA stage when calculating a within-group sum of squares and cross products matrix which turns out to be singular if the input variables are linearly correlated. This PCA stage is unsupervised, and it uses the covariance matrix derived from all of the calculated feature vectors.

This decorrelated version of data leaves the important information about cell differences whilst removing data correlations. This procedure transforms the original basis vectors in the feature space into specific new basis vectors that are rotated with respect to the original basis vectors. The original dataset is transformed in such a way that the features become maximally decorrelated. This dataset still retains the meaning and the information content of the original features. Further data analysis proceeds in this new PCA-decorrelated space.

The example quantitative feature may be a spatial average of channel intensity for each cell divided by the cell area for each cell. To calculate this example quantitative feature, the pixels corresponding to a specific cell may be identified by a procedure known as “cell segmentation”, and the corresponding autofluorescence signal measured within those specific pixels is added across each pixel, separately for each pair of the excitation spectral channel and emission spectral channel used. The area of the cell is calculated by counting the pixels belonging to that cell. The two values thus obtained are then divided producing average autofluorescence intensity in that cell in this pair of excitation/emission spectral channels. In other examples, the quantitative feature may be a mean, median, variance, kurtosis, or other Haralick feature calculated for each channel separately, or various derivative features combining individual channel features, such as channel ratios (the ratio of average cell intensities in two different channels) derived from the measured multispectral or hyperspectral autofluorescence images for that specific cell. These features are separately calculated in all pairs of excitation/emission spectral channels. The features may be defined in a way that reflects specific biology of the cell under investigation, for example average content of specific fluorophores of relevance to a particular cellular pathway (such as bound NADH, free NADH, FAD, flavins, cytochrome C and many others), or for example characteristics of the mitochondria such as their perinuclear location or specific shape, or shape distribution. Then the average channel intensity for each cell, or any other quantitative channel feature or the set of features for each cell may be assigned a type or class label such as “healthy”, “reference” or “diseased” or alternatives.

It may also be possible to use non-cellular features for classification, in particular pixel features, where features for each cell are not calculated, but use the pixel data for each of the classes or groups. For example, one could use raw pixel autofluorescence signals or secondary features such as width of pixel autofluorescence signal distributions in each of the channels as quantitative features. Alternatively, one could use quantitative features which incorporate some cellular identifications but do not imply taking cellular averages, for example raw pixel autofluorescence signals, only from the largest 10% of cells.

The feature space may then be transformed to a “new vector space” to optimally present cell group separation.

In some examples, the new vector space is produced by Linear Discriminant Analysis (see, for example, J. Ye, “Characterisation of a family of algorithms for generalized discriminant analysis on undersampled problems”, J. Mach. Learn. Res. 6 (2005) 483-502). The “new” vector space means a vector space whose set of basis vectors differs from the set of basis vectors of the original feature space. In other examples, the new vector space may be produced by alternative methods, including rotation under subjective manual control.

This means that the set of quantitative features thus obtained for each cell can then be projected onto a vector space that optimally discriminates or separates the data based on this class assignment. This projection may be done by using LDA. The dimensionality of this new space and hence the number of new canonical variables is P−1, where P is the number of unique classes assigned to the data (for example, if trying to separate two classes “healthy” and “sick” then P=2 and the projection is onto a 1 dimensional space). The data are projected onto specific directions determined by LDA, these directions based on the actual cell data. The coordinates of each cell are now expressed in terms of these canonical variables, sometimes case called “spectral variables”, and reflect the distance measured along these specific directions.

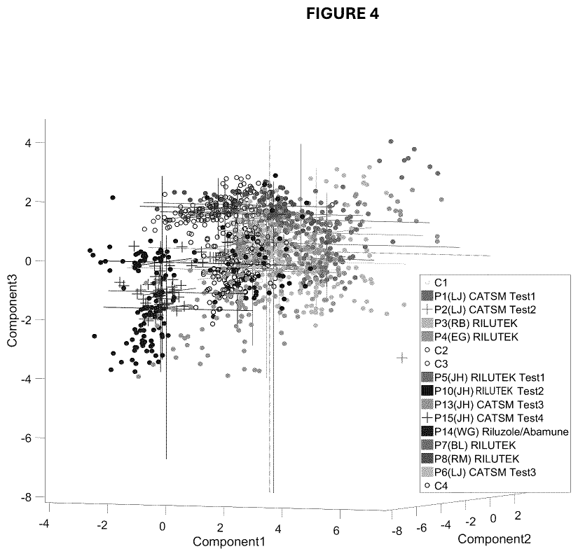

FIGS. 4 to 6 illustrate analyses of data collected from different patients and control individuals. The table below provides the labels used for the data in FIGS. 4 to 6.

| Testing | Testing | | |
|---|---|---|---|
| notation | notation used | Patient notation | |
| used in | (Measurement | used in FIGS. | |
| FIG. 4 | number) | 5 and 6 | Treatment drugs |
| Control 1 (C1) | 1 | Healthy control | None |
| P1 | 2 | ALS patient 1, T0 | Copper ATSM |
| | | | (CuATSM) |
| P2 | 3 | ALS patient 1 T1 | Copper ATSM |
| | | | (CuATSM) |
| P3 | 4 | One of eight | Riluzole |
| | | other patients | (brand name— |
| | | (Patients 3-10) | Rilutek ™) |
| P4 | 5 | One of eight | Riluzole |
| | | other patients | (brand name— |
| | | (Patients 3-10) | Rilutek ™) |
| P5 | 6 | ALS patient 2 T0 | Riluzole |
| | | | (brand name— |
| | | | Rilutek ™) |
| P6 | 7 | ALS patient 1 T2 | Copper ATSM |
| | | | (CuATSM) |
| P7 | 8 | One of eight | Riluzole |
| | | other patients | (brand name— |
| | | (Patients 3-10) | Rilutek ™) |
| P8 | 9 | One of eight | Riluzole |
| | | other patients | (brand name— |
| | | (Patients 3-10) | Rilutek ™) |
| P9 | 10 | One of eight | Riluzole |
| | | other patients | (brand name— |
| | | (Patients 3-10) | Rilutek ™) |
| Control 2 (C2) | 11 | Healthy control | None |
| Control 3 (C3) | 12 | Healthy control | None |
| P10 | 13 | ALS patient 2 T1 | Riluzole |
| | | | (brand name— |
| | | | Rilutek ™) |
| P11 | 14 | One of eight | Riluzole |
| | | other patients | (brand name— |
| | | (Patients 3-10) | Rilutek ™) |
| P12 | 15 | One of eight | Riluzole |
| | | other patients | (brand name— |
| | | (Patients 3-10) | Rilutek ™) |
| P13 | 16 | ALS patient 2 T2 | Copper ATSM |
| | | | (CuATSM) |
| P14 | 17 | One of eight | Riluzole/Abamune |
| | | other patients | |
| | | (Patients 3-10) | |
| Control 4 (C4) | 18 | Healthy control | None |
| P15 | 19 | ALS patient 2 T3 | Copper ATSM |
| | | | (CuATSM) |

Referring to FIG. 4, there is illustrated a projection of selected feature data derived from measured autofluorescence features of cells from individuals in a study group. The legend assigns a particular label to each individual, with C1 C4 corresponding to controls and P1 P15 corresponding to patients. In the legend, CATSM stands for CuATSM, Rilutek stands for Riluzole, and Abamune is an HIV medication. The supervised projection of the data onto a new vector space is designed to best discriminate between a first group of data (C1, C2, C4, P2, P6) and a second group of data (P1, P3, P4).

In FIG. 4, all controls C1, C2, C3 and C4 are clustering left of the untreated patient cells. CATSM and Riluzole/Abamune responses appear in the control space or to the left hand side. P1 patient data moves from the patient space (for Test 1) to the control space (for Test 2 and Test 3). P6 Test 3 is also close to the control space, as expected for treated cells. A nice progression is visible for patient JH from P5 (JH) Rilutek Test1 in the patient space, progressively moving left (and less component 2) into the control/treated space P5 (JH) CATSM Test2, P5 (JH) CATSM Test3, to P5 (JH) CATSM Test4.

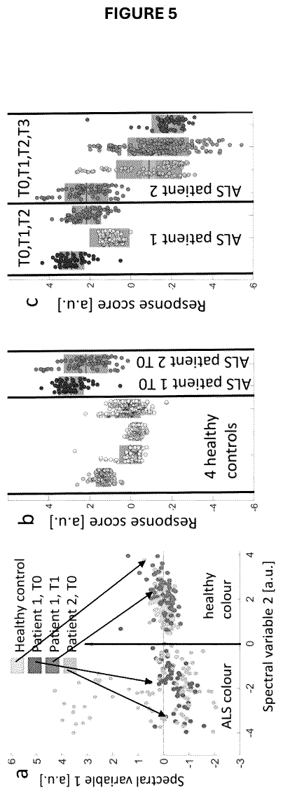

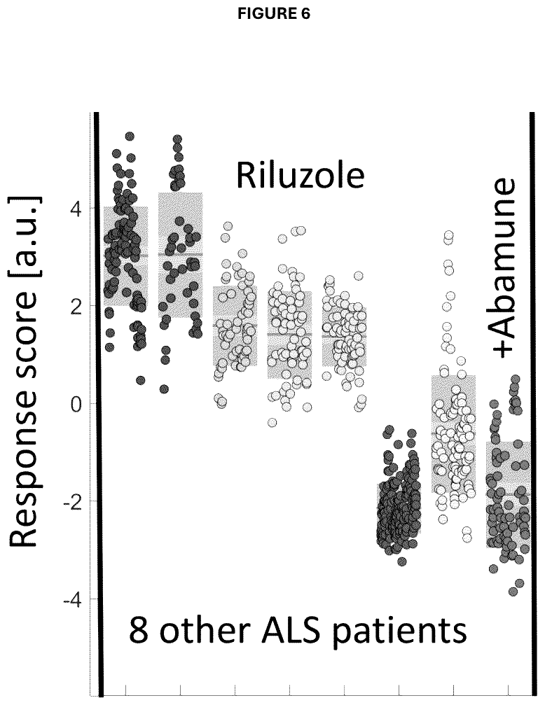

The concept of the analysis presented in FIGS. 5 and 6 was to convert multidimensional vectors representing each cell in each patient into one-dimensional “response vectors”. In general, these vectors may be positioned at any location within the data space in order to best fit an overall hypothesis, particularly to allow for patient factors. In some examples, the conversion of multidimensional vectors into one dimensional response vectors may carried out by a projection onto a hyperplane in the case of a more sophisticated model.

In the particular implementation presented here the “response vectors” were obtained using the following procedure. First, a discriminatory model was developed using a subset of the data based on two groups, the first group comprising the controls C1 C4 and two sets of patient data P2 and P6, while the second group comprising P1, P3, and P4. Second, all data from FIG. 4 was projected on the direction joining the centres of the two cell data clusters for the two groups defined above and a one-dimensional response vector was determined by a projection of each original cell vector from FIG. 4. These one-dimensional response vectors for all cells were presented in a box plot format (each symbol indicates a response vector for a different cell).

The box plots in FIGS. 5 and 6 comprise rectangles with three shades of grey. The thin line in the centre of the rectangle has the darkest shade of grey and represents the median value; the two stripes immediately adjacent to the centre line have the lightest shade of grey and represent the 95% confidence region (1.96×SEM); and the two stripes nearest to the ends of the rectangle have an intermediate shade of grey and represent the first standard deviation. If the light-grey regions of a cluster are not horizontally overlapping, one can be confident of having measured a difference. The values in the plot were shifted by a value of 1.3358, just for sake of placing the controls about zero, although this is an arbitrary shift and, in other examples, the values may be shifted by other amounts. Clusters may be classified according to their mean values: high values may be attributed to “diseased and unresponsive” state, intermediate values may be attributed to “normal”, and low values compared to normal may be interpreted as “drug affected”.

FIGS. 5 and 6 represent specific selections of individuals from the study cohorts from FIG. 4.

FIG. 5(a) shows the clustering of monocytes from a control healthy individual and from two ALS patients (Patient 1 and Patient 2). The sample from Patient 1 was measured twice, before (T0) and three weeks after (T1) the patient started treatment with CuATSM, where the mechanism of action is believed to correct specific redox abnormalities in ALS. The data show that the clusters of cells of both ALS individuals are clearly separated from the healthy control (also shown in FIG. 4). Strikingly, after commencing treatment, the cells of Patient 1 normalised towards the healthy cells. A one-dimensional projection of results for all patients (“response score”) shows that the group of four healthy controls form a tight cluster (shown in FIG. 5(b)). Similarly, close values of response scores were observed in the best proxies available for the untreated ALS group, namely ALS patients not responding to previous treatments (Patient 1 and 2, T0) and chosen to commence treatment with CuATSM (see FIG. 5(b)). Using these two groups, the difference of group averages (D) and the pooled group variance(S) was calculated. The ratio of D/S obtained was 4.23, indicating a significant separation consistent with the possibility of ALS diagnostics.

FIG. 5(c) shows results in patients 1 and 2 before (T0) and longitudinal testing over the course of CuATSM treatment (T1 T3). Patients 1 and 2 show statistically strong drug responses from time T1 onwards, especially Patient 2 whose responses followed a downward trend.

Eight other patients, most of whom were on Riluzole, only show a much wider range of response scores than healthy controls (shown in FIG. 6), which may be attributed to varying reactions to therapy.

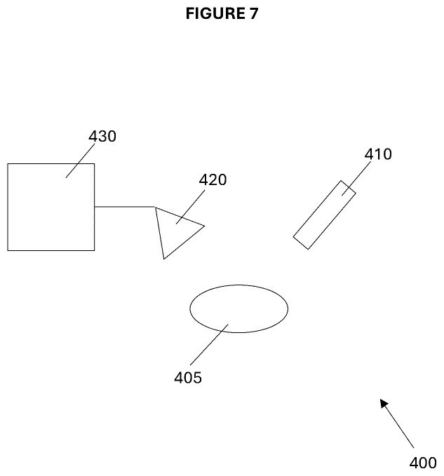

Referring to FIG. 7, there is illustrated an example system 400 for spectral analysis of cells 405. System 400 includes a light source 410 for stimulating, or exciting, cells 405 by irradiation with electromagnetic radiation having one or more wavelengths in an excitation spectral channel. System 400 further includes a detector 420 for detecting autofluorescence of cells 405. System further includes a processing system 430 configured to analyse spectral characteristics of autofluorescence of cells 405.

Light source 410 may include a laser or a light-emitting diode (LED). In some examples, light source 410 includes two or more lasers, or two or more LEDs. In some examples, light source 410 includes solid state excitation sources. In other examples, light source 410 includes any other source of electromagnetic radiation. In some examples, light source 410 is a tunable light source, having a tunable output wavelength.

In some examples, light source 410 is a broadband light source having a radiation output including multiple wavelengths, within a wavelength range or spectral channel. In some examples, system 400 further includes one or more optical spectral filters coupled to the output of light source 410 to spectrally shape, or to spectrally discriminate, the output of light source 410. In such examples, proper selection of the filter characteristics enables spectral shaping of the excitation spectral channel for stimulating cells 405. In some examples, system 400 further includes a device or system to determine excitation wavelength ranges.

Detector 420 may include a photodetector, such as a photodiode or a phototransistor. In some examples, detector 420 is an array detector. In some examples, detector 420 is an array detector including two or more photodetectors. In other examples, detector 420 is any other type of photodetector or light sensor, such as a camera (e.g. ANDOR iXon EMCCD cameras). In some examples, system 400 further includes an optical filter (not shown) for filtering radiation input into detector 420.

In some examples, system 400 further includes a microscope (not shown) for facilitating the detection of fluorescence of cells 405 by detector 420. In some examples, detector 420 is coupled to an optical output, such as an ocular lens, of the microscope. Examples of suitable microscopes include, but are not limited to, Olympus IX71 inverted epifluorescence microscope with UV enhanced objectives.

Preferably, though not necessarily, detector 420 is connected to processing system 430. In some examples, the connection between detector 420 and processing system 430 is a wired connection (e.g. via one or more cables). In other examples, the connection between detector 420 and processing system 430 is a wireless connection. In some examples, data collected by detector 420 is input into processing system 430.

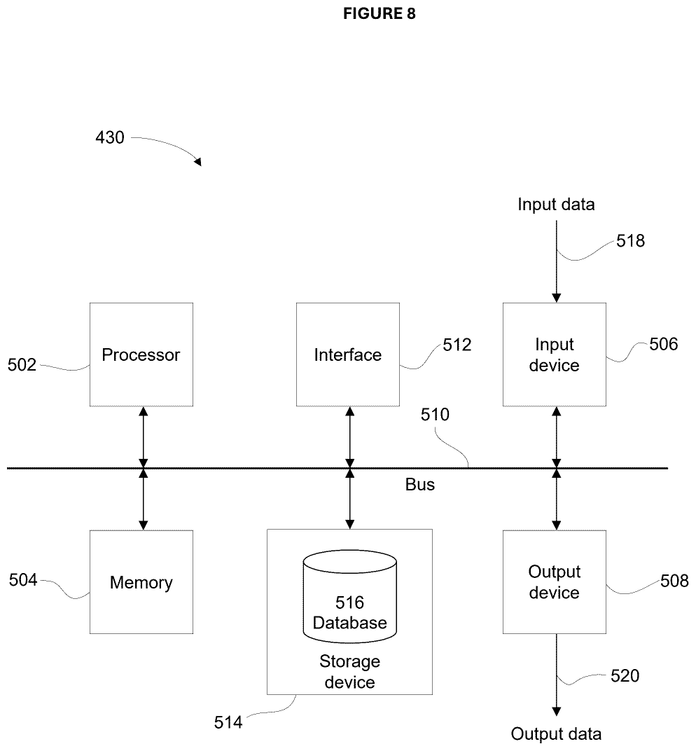

Referring to FIG. 8, there is illustrated an example processing system 430 of system 400. In particular, the processing system 430 generally includes at least one processor 502, or processing unit or plurality of processors, memory 504, at least one input device 506 and at least one output device 508, coupled together via a bus or group of buses 510. In certain embodiments, input device 506 and output device 508 could be the same device. An interface 512 can also be provided for coupling the processing system 430 to one or more peripheral devices, for example interface 512 could be a PCI card or PC card. At least one storage device 514 which houses at least one database 516 can also be provided. The memory 504 can be any form of memory device, for example, volatile or non-volatile memory, solid state storage devices, magnetic devices, etc. The processor 502 could include more than one distinct processing device, for example to handle different functions within the processing system 430.

Input device 506 receives input data 518 and can include, for example, a keyboard, a pointer device such as a pen-like device or a mouse, audio receiving device for voice controlled activation such as a microphone, data receiver or antenna such as a modem or wireless data adaptor, data acquisition card, etc. Input data 518 could come from different sources, for example keyboard instructions in conjunction with data received via a network. Output device 508 produces or generates output data 520 and can include, for example, a display device or monitor in which case output data 520 is visual, a printer in which case output data 520 is printed, a port for example a USB port, a peripheral component adaptor, a data transmitter or antenna such as a modem or wireless network adaptor, etc. Output data 520 could be distinct and derived from different output devices, for example a visual display on a monitor in conjunction with data transmitted to a network. A user could view data output, or an interpretation of the data output, on, for example, a monitor or using a printer. The storage device 514 can be any form of data or information storage means, for example, volatile or non-volatile memory, solid state storage devices, magnetic devices, etc.

In use, the processing system 430 is adapted to allow data or information to be stored in and/or retrieved from, via wired or wireless communication means, the at least one database 516. The interface 512 may allow wired and/or wireless communication between the processing unit 502 and peripheral components that may serve a specialised purpose. The processor 502 receives instructions as input data 518 via input device 506 and can display processed results or other output to a user by utilising output device 508. More than one input device 506 and/or output device 508 can be provided. It should be appreciated that the processing system 430 may be any form of terminal, server, specialised hardware, or the like.

In some examples, processing system 430 is further configured to perform specific image pre-processing (Steps A-J). Processing system 430 is further configured to calculate, for the imaged cells, a set of quantitative features of the measured autofluorescence using the information from some or all pairs of excitation and emission channels where images have been taken. Processing system 430 is further configured to decorrelate (by PCA) the quantitative features, and to project, for each cell, the quantitative cellular feature vectors (one for each segmented cell) onto a new vector space which is suitably chosen.

It will be appreciated by persons skilled in the art that numerous variations and/or modifications may be made to the disclosure without departing from the spirit or scope of the invention as broadly described. The present embodiments are, therefore, to be considered in all respects as illustrative and not restrictive.

Optional embodiments may also be said to broadly include the parts, elements, steps and/or features referred to or indicated herein, individually or in any combination of two or more of the parts, elements, steps and/or features, and wherein specific integers are mentioned which have known equivalents in the art to which the invention relates, such known equivalents are deemed to be incorporated herein as if individually set forth.

All publications mentioned in this specification are herein incorporated by reference. The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure will now be described with reference to the following specific examples, which should not be construed as in any way limiting the scope of the disclosure.

### EXAMPLES

### Example 1—Cell Isolation and Sample Preparation

### Blood Collection and Processing

Blood samples were collected using Vacutainer® CPT™ Cell Preparation Tube (BD Australia, catalogue: 362760). 4 mls of whole blood from controls and MND patients were collected into the CPT tube via veinpuncture technique, using the standard technique for BD Vacutainer® Evacuated blood collection tubes. After collection, the tube/blood samples were centrifuged at room temperature (18-25° C.) in a horizontal rotor centrifuge for a minimum of 20 minutes at 1500 to 1800 RCF. After centrifugation, mononuclear cells and platelets located in a whitish layer (Buffy layer just under the plasma layer) were collected using a Pasteur pipette and transferred to a 15 mL size conical centrifuge tube.

### Monocyte Isolation

Monocytes were isolated from collected buffy layer using a Pan Monocyte Isolation Kit, human (Miltenyi Biotec, catalogue: 130-096-537). The Monocyte Isolation Kit is an indirect magnetic labelling system for the isolation of untouched monocytes from human peripheral blood mononuclear cells (PBMCs). Non-monocytes, i.e. T cells, NK cells, B cells, dendritic cells and basophils, are indirectly magnetically labeled using a cocktail of biotin-conjugated antibodies against CD3, CD7, CD16, CD19, CD56, CD123 and Glycophorin A, and Anti-Biotin MicroBeads. Isolation of highly pure unlabelled monocytes is achieved by depletion of the magnetically labeled cells.

The procedure of isolation included the following steps according kit instructions:
a) mixing 1 ml of collected buffy layer with 1 ml of buffer, and centrifugation at 300 g for 10 min, followed by aspiration of the supernatant;
b) resuspending the cell pellet in 30 μl of buffer per 10⁷ total cells;
c) adding 10 μL of FcR Blocking Reagent and 10 μL of Biotin-Antibody Cocktail per 10⁷ total cells, mixing well and incubating for 5 mins at 2-8° C.;
d) adding 30 μL of buffer, 20 μL of Anti-Biotin MicroBeads and 20 μL of CD61 platelets depletion microbeads per 10⁷ total cells, mixing well and incubating for 12 mins at 2-8° C.;
e) subsequent manual cell separation using a cell separation column placed in the magnetic field of a suitable MACS Separator; and
f) applying the cell suspension to the column and collecting the flow-through containing unlabelled cells, representing the enriched monocytes.

### Neutrophil Isolation

Neutrophils were isolated from whole blood using a MACSxpress Neutrophil isolation kit (Miltenyi Biotec, catalogue: 130-104-434). In this method, freshly drawn anticoagulated whole blood was used without density gradient centrifugation. Erythrocytes were aggregated and sedimented, while non-target cells were removed by immunomagnetic depletion with MACSxpress beads.

The procedure of isolation included the following steps according to kit instructions
a) reconstituting and preparing the lyophilized pellet of beads followed by preparation of final cocktail of beads by mixing appropriate volumes of reconstituted pellet and buffer;
b) processing 4 ml of whole blood by adding 1 ml of reconstituted pellet and 1 ml of buffer in a 15 ml tube and mixing by gently pipetting up and down 3-4 times, followed by incubation for 5 mins at room temperature using the MACSmix Tube Rotator on permanent run speed of approximately 12 rpm;
c) placing the open tube in the magnetic field in in the MACSxpress separator for 15 mins (the magnetically labelled cells will adhere to the wall while the aggregated erythrocytes sediment to the bottom); and
d) collecting the supernatant (neutrophils) with the tube still inside the MACSxpress separator, by carefully moving the pipette tip top to bottom down the front wall of the tube.

### Sample Preparation for Hyperspectral Imaging

Monocyte suspensions in Hanks Balanced Salt Solution were maintained at 2-8° C. and imaged immediately. Prior to imaging, 35 mm dishes with cover glass bottoms were coated with 0.01% Poly-L-lysine solution (Sigma Aldrich, catalogue; P4707) to assist cell attachment to the surface. 300 μL of monocyte suspension was transferred into imaging dishes to facilitate imaging.

### Example 2—System for Hyperspectral/Multispectral Imaging of Cellular Autofluorescence

In one example embodiment, images of live cells were obtained by an Andor IXON camera under illumination at a number of selected bands of excitation wavelengths (centred at 334, 365, 385, 395, 405, 415, 425, 435, 455, 475, 495 nm, each about 10 nm wide). The illumination was supplied by a plurality of light-emitting diodes (LED). The emission was measured with a 532 nm long pass dichroic mirror together with a 587 nm bandpass filter (35 nm bandwidth), in the range 570 nm to 605 nm. The list of pairs of excitation/emission channels in given in paragraph [0139].

The above example selection of pairs of excitation and emission channels with optimised exposure times typically enables the capture of cellular images with sufficient signal to noise ratio for accurate unmixing of multiple cellular fluorophores, and the calculation of cellular features. The list of possible unmixed fluorophores includes but is not limited to free and bound nicotinamide adenine dinucleotide (NADH) whose spectra have tails in the 570 nm to 605 nm range, however these compounds produce a significant proportion of the autofluorescence signal at 334 nm excitation wavelength. Optical powers at the objective ranged from 0.1 μW at 334 nm excitation to 102 ρW at 475 nm, but typically are in the order of a few microwatts. A “background” reference image of a culture dish with a medium is also taken and subtracted from all images with cells (Step A). The time of imaging is adjusted for each channel to obtain a well-exposed image, without saturated areas and not too dark. In a well-exposed image, an average saturation between 40% to 60% of the available maximum is considered satisfactory.

### Example 3—Hyperspectral Imaging of Cellular Autofluorescence

### Hyperspectral Hardware Setup

In one example embodiment, a fluorescence microscope (Olympus iX71™) was used with a 40× water U12™ series objective, with the wide transmission in UV range. Selected bands of excitation wavelengths (centred at 334, 365, 375, 385, 395, 405, 415, 425, 435, 455, 475, 495 nm, each about 10 nm wide) were used to excite cell autofluorescence. Three epifluorescence filter cubes were available to measure single photon-excited emission of biological samples. With these twelve excitation sources and three filters, a total of 18 specific channels were created, as listed in the Table in paragraph [0139]. Optical powers at the objective ranged from 0.01 ρW (at 495 nm excitation with 587 nm emission, channel 15) to 42.8 μW (at 475 nm excitation with 587 nm emission, channel 14). The excitation sources were coupled by an optical fibre bundle with a 5 mm fused silica hexagonal homogenizer. The excitation sources produced a reasonably flat approximately Gaussian distribution of illumination over the sample plane, whose flatness was further corrected digitally. All images were captured by Andor iXON™ camera (EMCCD, iXON 885 DU, Andor Technology Ltd., UK) operated below −65° C. to reduce sensor-induced noise. Some of the underpinning noise mechanisms depended on illumination level and they could not be reduced by sensor cooling. The sensor size was 1002×1004 pixels.

### Image Pre-Processing

The pre-processing steps include taking first set of reference images (Step A) image equalization (Step B), primary denoising with removal of undetectable pixels (Step C) and outliers (spikes or dips) (Step D), image smoothing (Step E), removing background fluorescence (Step F) measurement of calibration fluid (Step G), background illumination flattening (Step H), measurement of second set of reference images and spectra (Step I), and cell segmentation (Step J). All this was carried out without changing the mathematical structure of the dataset. The pixel identification (image number, pixel coordinates, spectral channel etc.) were separately retained for the reconstruction of two-dimensional fluorophore abundance maps.

### Step A—Taking First Set of Reference Images

At the beginning of each experiment, a first set of reference images including water, and dark images were taken using the hyperspectral microscope system. These reference images were then used to pre-process the sample images.

### Step B—Image Equalisation

In the image equalisation procedure, the intensity count at every channel was converted into the units of photons per pixel per second (PPS). This calculation helped to standardize images taken with different acquisition parameters, most notably electron-multiplication (EM) gain, and acquisition time. For the Andor iXON™ camera used, the sample signal expressed in terms of photon per second, y_{raw[PPS]}, was given by:

$${y}_{raw [ PPS ]} ( k , i ) = \frac{( {y}_{raw [ digital ]} ( k , i ) - {BO}_{[ digital ]} ) \times se}{{G}_{EM} \times QE \times {t}_{exp}}$$

where y_{k,i[digital]} denoted the measured digital counts (in the range 0-2¹⁴). The bias offset (B0_{[digital]}) used in the setup was 100 counts. The camera sensitivity (se) for the readout rate of 13 MHz was 0.89. The EM gain (G_{EM}) and exposure time (tₑₓₚ) were adjusted by the operator taking into account the sample signals, and they were generally different in different channels. The quantum efficiency (QE) of the camera sensor was also different for different channels.

### Step C—Removing Undetectable Pixels

Two sets of dark images (acquired, respectively, with the microscope shutter open and closed) were taken to remove the undetectable pixels. Such undetectable pixels could be due to light blockages (e.g. by dust) between the sensor and the sample plane, or inactive camera pixels. The average of these two dark images was subtracted from all sample, water and calibration images, to correct for any pixels that were unresponsive.

### Step D—Removing Outliers (Spikes)

Abnormal behaviour of sensor pixels in combination with high EM sensor gain may cause random sharp spikes or sharp dips in the image. To remove these outliers, a ‘threshold limiting window’ was scanned over all the images to locate these spikes or dips. Then these specific data points were replaced with the values interpolated from immediately adjacent nine pixels.

### Step E—Image Smoothing

The main sources of noise from EMCCD camera included illumination independent and illumination-dependent noise. The illumination independent noises (e.g. dark-current shot noise, readout noise etc.) were minimised by using low sensor temperature (below −65° C.). Illumination-dependent noise (e.g. photon shot noise, clock induced charge noise, EM gain register noise etc.) was considered as multiplicative temporal and spatial noise. The overall noise in autofluorescence images was a combination of illumination dependent noise which was approximately Poissonian, while the noise from the illumination independent sources could be modelled as a Gaussian noise. Gaussian noise was used in simulation as a proxy for the overall noise, because the Poisson's noise amplitude cannot be modified independently from the signal. A customised wavelet filter was used to remove the image noise for smoothing, which facilitated improved capture of spectral information from a signal compared with standard frequency spectra produced by Fourier analysis. The wavelets help to divide the signal into different scale components and thus these customized wavelet filters have proved to be a computationally efficient method of capturing textural information from filters or banks of filters with attractive attributes with potentially lossless coverage of the frequency spectrum.

### Step F—Removing Background Autofluorescence

The images were also affected by the unavoidable autofluorescence signals from the microscope slide, Petri dishes, dirt on sensors etc. These signals make additive contributions to all images. To remove these contributions, two hyperspectral images were taken of water in the petri dish used for imaging. The smoothed average of these two images is denoted by B (k, i). This smoothed average image, different for each channel, was subtracted from each sample image in this specific channel.

### Step G—Measurements of Calibration Fluid Images

The microscope system was calibrated by taking hyperspectral images of a “calibration fluid”, which in these experiments was a mixture of 30 μM NADH (quantum yield 0.019) and 5 μM riboflavin (quantum yield 0.24). Its composition was adjusted so that the spectrum of the calibration fluid had non-zero response across all the spectral channels. The smoothed image of the calibration fluid is denoted by C_{raw}(k, i).

### Step H—Image Flattening

Finally, the raw sample image, y_{raw}(k, i), was corrected by using the averaged and smoothed background image B(k, i). Furthermore, the smoothed image of the calibration fluid was used to correct for the somewhat uneven (approximately Gaussian) illumination of the field of view. This was done by dividing the sample image in each channel (after subtracting of the smoothed water image) by the relevant smoothed image of the calibration fluid. These corrections are specified in the equation below:

$$y ( k , i ) = \frac{f ( k ) \times ( {y}_{raw} ( k , i ) - B ( k , i ) )}{{C}_{raw} ( k , i ) - B ( k , i )} .$$

### Step I—Measurement of a Second Set of Reference Images and Spectra

In the case when fluorophore unmixing is required, the relationship between standard fluorimetry and hyperspectral/multispectral microscopy of a set of reference pure fluorophore compounds must be obtained. The pure fluorophores are diluted to approximately physiological concentrations in the micromolar range. Their fluorescence spectra in the wavelength ranges corresponding to each pair of the excitation/emission channels are measured using standard fluorimetry and the same samples are then imaged using a multispectral/hyperspectral microscope, in all pairs of excitation/emission channels. The reference images and spectra are then utilised for fluorophore unmixing which may be carried out in the current context as per the publication “Statistically strong label-free quantitative identification of native fluorophores in a biological sample” by Saabah B. Mahbub, et al., Scientific Reports, volume 7, article number: 15792(2017). Fluorophore unmixing may or may not be required to identify quantitative features of relevance.

### Step J—Cell Segmentation

In order to calculate cellular features, cellular images needed to be segmented into individual cells. In this example, retina cells were selected manually with the overlaid DIC image. The normalized autofluorescence intensity in each cell was documented in the yₖᵢ matrix. The image number, pixel coordinates i and the spectral channel indices k were saved separately for the reconstruction of two-dimensional fluorophore abundance maps.

### List of Spectral Channels

The list of channels provided in the Table below was used for the analysis of the motor neurone disease cells shown in FIGS. 4 to 6.

| | | Emission | | Dichroic | | Quantum |
|---|---|---|---|---|---|---|
| Spectral | Excitation | wavelength | | mirror | Power at | efficiency |
| channel | wavelength | (bandwidth) | | long pass | objective | (QE) (unit |
| number | (±5 nm) | (nm) | | (nm) | (μW) | less) |
| 1 | 334 | 447 | (60) | 409 | 0.046 | 0.5 |
| 2 | 365 | 447 | (60) | 409 | 6.5 | 0.5 |
| 3 | 375 | 447 | (60) | 409 | 2.83 | 0.5 |
| 4 | 334 | 587 | (35) | 532 | 0.02 | 0.65 |
| 5 | 365 | 587 | (35) | 532 | 6.4 | 0.65 |
| 6 | 375 | 587 | (35) | 532 | 10.51 | 0.65 |
| 7 | 385 | 587 | (35) | 532 | 17.77 | 0.65 |
| 8 | 395 | 587 | (35) | 532 | 13.33 | 0.65 |
| 9 | 405 | 587 | (35) | 532 | 9.39 | 0.65 |
| 10 | 415 | 587 | (35) | 532 | 22.4 | 0.65 |
| 11 | 425 | 587 | (35) | 532 | 20.9 | 0.65 |
| 12 | 435 | 587 | (35) | 532 | 27.5 | 0.65 |
| 13 | 455 | 587 | (35) | 532 | 14.8 | 0.65 |
| 14 | 475 | 587 | (35) | 532 | 42.8 | 0.65 |
| 15 | 495 | 587 | (35) | 532 | 0.01 | 0.65 |
| 16 | 405 | 700 | (long pass) | 635 | 9.5 | 0.63 |
| 17 | 455 | 700 | (long pass) | 635 | 15.09 | 0.63 |
| 18 | 495 | 700 | (long pass) | 635 | 9.97 | 0.63 |

Specific quantitative features used for analysing spectral characteristics of autofluorescence of the MND cells which have allowed the separation of sick and healthy patients are the following six features: (1) Median of pixel intensity ratios between channels 12 and 16; (2) Mean of pixel intensity ratios between channels 6 and 20; (3) Variance of pixel intensity for channel 20; (4) Kurtosis of pixel intensity for channel 24; (5) Median of pixel intensity ratios between channels 3 and 18; and (6) Mean of pixel intensity ratios between channels 10 and 16. The channel numbers refer to Table in paragraph [0139].

In some cases it may be necessary to identify the distinguishing quantitative features de novo. This process involves calculating a set of potential quantitative features, taking various smaller subsets of these quantitative features, evaluating group/class separations for each of these subsets, and selecting those subsets that provide the largest possible group/class separations. It may be also possible to use artificial intelligence software to provide suitable smaller subsets of such quantitative features.

### Example 4-Principal Component Analysis (PCA)

Let X be the n by p data matrix of observed pixel spectra by wavelength, wherein n>>p. The data covariance matrix S can be expressed by

$$S = {X}^{T} ( I - \frac{1}{n} {e}_{n} {e}_{n}^{T} ) X$$

where eₗ denotes an l-th basis vector.

The principal component analysis of X may be obtained through the eigenvalue decomposition of nS:

where

$$S = V {Σ}^{2} {V}^{T}$$

where

$${Σ}^{2} = diag ( {σ}_{1}^{2} {σ}_{2}^{2} , … , {σ}_{p}^{2} ) ,$$

or the variance vector of each variable and V represents the orthogonal matrix of eigenvectors forming the basis vector of the new space onto which the data may be projected forming the new decorrelated variables Y.

$$Y = X \times V$$

### Example 5—Linear Discriminant Analysis (LDA)

Falling within the framework of supervised techniques, linear discriminant analysis looks to find the basis axes which maximise the Maximum Fisher distance of the projected data. This criterion is the ratio of the between-class scatter to the within-class scatter.

Here it is assumed that there are C pattern classes, ω₁, ω₂, . . . , ω_{c} in a pattern space of N dimensions, where: lᵢ is the number of samples in class i; xᵢⱼ denotes the j-th sample of class i; μᵢ is the mean vector of the samples in class i, where it is assumed that the mean value is the expected value μᵢ=E(xᵢⱼ|ωᵢ) of the population of class i; and μₒ is the expected value (mean) of the entire data set. Then the between-class scatter is defined as

$${S}_{b} = \frac{1}{M} {\sum }_{i = 1}^{C} {i}_{i} ( {μ}_{i} - {μ}_{o} ) {( {μ}_{i} - {μ}_{o} )}^{T}$$

and the within-class scatter as

$${S}_{b} = \frac{1}{M} {\sum }_{i = 1}^{C} {\sum }_{j = 1}^{{i}_{i}} ( {x}_{ij} - {μ}_{i} ) {( {x}_{ij} - {μ}_{i} )}^{T}$$

The classes or groups of observations can be chosen arbitrarily (for example, one may consider two classes: “cells from controls” and “cells from sick patients”).

An estimate of the class mean u; is obtained through calculating the class sample average, and similarly

$${m}_{i} = \frac{1}{{i}_{i}} {\sum }_{j = 1}^{{i}_{i}} {x}_{ij}$$

the average of all samples is used to estimate the expected value (mean) of the entire data set.

$${m}_{o} = \frac{1}{M} {\sum }_{i = 1}^{C} {\sum }_{j = 1}^{{i}_{i}} {x}_{ij}$$

The Fisher criterion sought to be maximised is expressed as

$${J}_{F} ( w ) = \frac{{w}^{T} {S}_{b} w}{{w}^{T} {S}_{w} w}$$

The eigenvectors w₁, w₂, . . . , w_{d} of S_{b}w=λS_{w}w form the new coordinate system. The corresponding eigenvalues indicate the ratio of between/within variance and, since the rank of S_{b} will be C−1, only that number of eigenvalues is non-zero.

Thus, one can be certain that projections of the input data into this space will provide a close to optimal class discrimination. There are now C−1 new canonical variables and for ease of visualization, it is useful to choose three classes of data in the discriminant analysis so that the result may be visualized in a 2D scatter plot. The pixel observations are further grouped according to their origin on a cell basis, and a statistical metric such as a group average may be calculated on each such group used to represent the group.

### Example 6—Haralick Features

Haralick features can be applied directly to autofluorescent images to obtain a broad description of image features structures. An effective method of obtaining a suite of textural features is by use of a co-occurrence matrix (see, for example, Haralick, R. M. and Shapiro, L. G., “Computer and robot vision”, Vol. 1, Addison-Wesley Longman Publishing Co, Inc, 1992). In an example embodiment, a computationally efficient means of calculating these features was used (see, for example, Clausi, D. A. & Zhao, Y. in Geoscience and Remote Sensing Symposium, 2002. IGARSS'02. 2002 IEEE International, Vol. 4 2453-2455 (IEEE, 2002)).

For each image, a grey level co-occurrence matrix, p_{d,θ} was defined. To obtain this matrix, pixel intensities were first divided into Ng=8 gray level bins. A selected pixel was then focused in on the image and the (binned) intensity of the pixel adjacent to it (at a distance of one pixel (d=1)) was considered, at a specific angle θ. If that adjacent pixel had the same grey intensity as the selected one, the value of co-occurrence was 1. These co-occurrence values were then added over all pixels in the entire image and the entire matrix was divided by the number of such co-occurrences. Each entry in this matrix was the probability p_{d,θ}(i, j) that a pixel with a quantised grey value i is adjacent to the pixel with a grey value j. There were four directions of adjacency with angles θ=0, 45, 90 and 135 degrees. The co-occurrence matrices were then four 8 by 8 arrays p_{d,θ} (θ=0, 45, 90, and) 135°.

Further, Haralick features may be generated with all four co-occurrence matrices thus obtained for the image and the maximum value of a feature thus obtained may be used as the final feature.

### Example 7—Kurtosis Feature

A sample estimate was used for the kurtosis. The sample estimate, k, was given by:

$$k = \frac{\frac{1}{n} {\sum }_{i = 1}^{n} {( {x}_{i} - \overline{x} )}^{4}}{{[ \frac{1}{n} {\sum }_{i = 1}^{n} {( {x}_{i} - \overline{x} )}^{2} ]}^{2}} - 3$$

Here, n is the total number of pixels in the image and xᵢ are pixel intensity values in an image.

### Example 8—Hyperspectral Autofluorescence Imaging of Monocytes in Motor Neuron Disease

Monocyte suspensions were prepared according to Example 1 from three subjects known to have a motor neuron disease and from a negative control (a subject known not to have a motor neuron disease). A monocyte suspension was also prepared from one of the motor neuron disease sufferers following administration of a therapeutic treatment for ALS (Cull(atsm)).

Hyperspectral autofluorescence imaging of these monocyte suspensions was carried out as described in the above examples. A scatter plot of cell spectra is shown in FIG. 10 in which the axes represent the directions onto which the cellular data have been projected by LDA. The LDA clearly separates cells from subjects with a motor neuron disease from the cells of the control subject. Moreover, the cells isolated from the subject with motor neuron disease following the administration of therapy are clearly distinguishable from the cells from the same subject prior to therapy.

1. A method for selecting a subject for treatment for a neurodegenerative disease, comprising:
obtaining from a subject a sample comprising at least one live blood cell and optionally isolating at least one live blood cell from the sample;
generating one or more multispectral or hyperspectral images of the at least one cell, and analysing spectral characteristics of autofluorescence from the at least one cell, to diagnose a neurodegenerative disease; and
selecting a subject, identified in (a) as having a neurodegenerative disease, for treatment for said disease.

2. The method according to claim 1, wherein the spectral characteristics of autofluorescence are compared to spectral characteristics of autofluorescence from a cell(s) derived from one or more reference samples known to be free of the neurodegenerative disease.

3. The method according to claim 1, wherein the at least one blood cell is a peripheral mononuclear blood cell.

4. The method according to claim 3, wherein the peripheral mononuclear blood cell is a monocyte.

5. The method according to claim 1, wherein a suspension comprising the at least one blood cell is subjected to the multispectral or hyperspectral autofluorescence imaging.

6. The method according to claim 1, wherein the sample comprising the at least one live blood cell is obtained from venous blood.

7. The method according to claim 1, wherein the at least one live cell is isolated by negative selection.

8. The method according to claim 1, wherein the one or more multispectral or hyperspectral images are generated by multispectral or hyperspectral microscopy.

9. The method according to claim 1, wherein the generating one or more multispectral or hyperspectral images includes stimulating the at least one cell by irradiation with electromagnetic radiation having one or more wavelengths in an excitation spectral channel and detecting autofluorescence of the at least one cell in an emission spectral channel.

10. The method according to claim 9, wherein the generating one or more multispectral or hyperspectral images is repeated for each pair of excitation spectral channel and emission spectral channel in a set of spectral channel pairs.

11. The method according to claim 9, wherein the emission spectral channel differs from the excitation spectral channel.

12. The method according to claim 1, wherein analysing spectral characteristics of autofluorescence from the cells comprises:
performing image pre-processing;
calculating, for each cell, quantitative features of measured autofluorescence;
removing correlations between the calculated quantitative features of different cells; and
projecting, for each cell, the quantitative features of the measured autofluorescence onto a new vector space.

13. The method according to claim 12, wherein the removing correlations uses Principal Component Analysis (PCA).

14. The method according to claim 12, wherein the new vector space is produced by Linear Discriminant Analysis (LDA).

15. The method according to claim 1, wherein the neurodegenerative disease is a motor neuron disease.

16. A method for monitoring a response of a subject to a therapeutic treatment for a neurodegenerative disease, and/or for monitoring an efficacy of a therapeutic treatment, the method comprising:
(a) obtaining from a subject a first sample before or after commencement of therapeutic treatment, wherein the first sample comprises at least one live blood cell, and optionally isolating at least one live blood cell from the sample;
(b) generating one or more multispectral or hyperspectral images of the at least one cell from the first sample, and analysing spectral characteristics of autofluorescence from the at least one cell;
(c) obtaining from the same subject a second sample at a time point after commencement of treatment and after the first sample is obtained, wherein the second sample comprises at least one live blood cell, and optionally isolating at least one live blood cell from the sample;
(d) generating one or more multispectral or hyperspectral images of the at least one cell from the second sample, and analysing spectral characteristics of autofluorescence from the at least one cell; and
(e) comparing said spectral characteristics of cells from the first and second samples, wherein the comparison between said spectral characteristics between the at least one cell from the first sample and the at least one cell from the second sample is indicative of whether or not the subject is responding to the therapeutic treatment and/or whether or not the therapeutic treatment is effective.

17. The method according to claim 16, further comprising obtaining and executing steps in respect of a third or subsequent sample.

18. A system configured to aid in detection or diagnosis of a neurodegenerative disease, the system comprising: a light source for stimulating live blood cells by irradiation with electromagnetic radiation having one or more wavelengths in an excitation spectral channel; a detector for detecting autofluorescence of the cells; and a processing system configured to analyse spectral characteristics of the autofluorescence of the cells, and optionally to provide a diagnostic prediction with respect to a subject.

19. The system according to claim 18, wherein the processing system is further configured to:
perform image pre-processing;
calculate, for each cell, quantitative features of detected autofluorescence;
remove correlations between the calculated quantitative features of different cells; and
project, for each cell, quantitative features of the detected autofluorescence onto a new vector space.
